(19) United States
(12) Patent Application Publication
(10) Pub. No.: US 2008/0234283 A1
(43) Pub. Date: Sep. 25, 2008

(54) Glutathione peroxidase mimetics for treatment of neurodegenerative, pulmonary and inflammatory diseases

(76) Inventor: Noah Berkowitz, New Rochelle, NY (US)

Correspondence Address: Pearl Cohen Zedek Latzer, LLP, 1500 Broadway, 12th Floor, New York, NY, 10036, US

(21) Appl. No.: 12/071,271

(22) Filed: Feb. 19, 2008

Related U.S. Application Data
(60) Provisional application No. 60/901,677, filed on Feb. 16, 2007.

(51) Int. Cl. A61K 31/33; A61K 31/395; A61K 31/335; A61K 31/4523; A61K 31/496; A61P 11/00; A61P 29/00; A61P 19/02; A61P 35/00; A61P 25/00; A61P 25/28

(52) U.S. Cl. 514/252.13; 514/183; 514/473; 514/462; 514/326

(57) ABSTRACT

This invention relates to compositions and methods for the treatment of neurodegenerative, pulmonary and inflammatory diseases described herein with glutathione peroxidase and its mimetics.

### CROSS-REFERENCE TO RELATED APPLICATION

This application claims priority under 35 U.S.C. §119(e) to U.S. provisional patent application Ser. No. 60/901,677, filed Feb. 16, 2007, and is incorporated herein by reference in its entirety.

### FIELD OF INVENTION

This invention is directed to methods for the treatment of neurodegenerative, pulmonary and inflammatory diseases with glutathione peroxidase and its mimetics.

### BACKGROUND OF THE INVENTION

Neurodegenerative diseases, have a strongly debilitating impact on patients' life aswell as their families. Furthermore, these diseases constitute an enormous health, social, and economic burden. Alzheimers Disease (AD) is the most common neurodegenerative disease, accounting for about 70% of all dementia cases, and it is probably the most devastating age-related neurodegenerative condition, affecting about 10% of the population over 65 years of age and up to 45% over age 85. Presently, this amounts to an estimated 12 million cases in the US, Europe, and Japan. This situation will inevitably worsen with the demographic changes due to aging of the epopulation resulting in longer life expectancy and lower birth rate in developed countries.

Diseases and disorders of the pulmonary system are among the leading causes of acute and chronic illness in the world. Pulmonary diseases or disorders may be organized into various categories, including, for example, breathing rhythm disorders, obstructive diseases, restrictive diseases, infectious diseases, pulmonary vasculature disorders, pleural cavity disorders, and others. Pulmonary dysfunction may involve symptoms such as apnea, dyspnea, changes in blood or respiratory gases, symptomatic respiratory sounds, e.g., coughing, wheezing, respiratory insufficiency, and/or general degradation of pulmonary function, among other symptoms. Obstructive pulmonary diseases can be associated with a decrease in the total volume of exhaled airflow caused by a narrowing or blockage of the airways. Examples of obstructive pulmonary diseases include asthma, emphysema and bronchitis. Chronic obstructive pulmonary disease (COPD) refers to chronic lung diseases that result in blocked airflow in the lungs. Chronic obstructive pulmonary disease may develop over many years, typically from exposure to cigarette smoke, pollution, or other irritants. Over time, the elasticity of the lung tissue is lost, the lung's air sacs may collapse, the lungs may become distended, partially clogged with mucus, and/or lose the ability to expand and contract normally. As the disease progresses, breathing becomes labored, and the patient grows progressively weaker. Many people with COPD concurrently have both emphysema and chronic bronchitis. Pulmonary disease may be caused by infectious agents such as viral and/or bacterial agents. Examples of infectious pulmonary diseases include pneumonia, tuberculosis, and bronchiectasis. Non-infectious pulmonary diseases include lung cancer and adult respiratory distress syndrome (ARDS), for example.

Inflammatory diseases, whether of a chronic or acute nature, represent a substantial problem in the healthcare industry. Briefly, chronic inflammation is considered to be inflammation of a prolonged duration (weeks or months) in which active inflammation, tissue destruction and attempts at healing are proceeding simultaneously. Although chronic inflammation can follow an acute inflammatory episode, it can also begin as an insidious process that progresses with time, for example, as a result of a persistent infection (e.g., tuberculosis, syphilis, fungal infection) which causes a delayed hypersensitivity reaction, prolonged exposure to endogenous (e.g., elevated plasma lipids) or exogenous (e.g., silica, asbestos, cigarette tar, industrial metals, surgical sutures) toxins, or, autoimmune reactions against the body's own tissues (e.g., rheumatoid arthritis, systemic lupus erythematosus, multiple sclerosis, psoriasis). Chronic inflammatory diseases therefore, include many common medical conditions such as rheumatoid arthritis, restenosis, psoriasis, multiple sclerosis, surgical adhesions, tuberculosis, chronic inflammatory lung diseases (e.g., asthma, pneumoconiosis, chronic obstructive pulmonary disease, nasal polyps and pulmonary fibrosis), periodontal disease (i.e., periodontitis) and polycystic kidney disease.

There is a need in the art for reliable therapy for patients suffering from neurodegenerative, pulmonary and inflammatory diseases.

### SUMMARY OF THE INVENTION

In another embodiment, the invention provides a method of treating a neurodegenerative disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In one embodiment, the invention provides a method of inhibiting or suppressing a neurodegenerative disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In another embodiment, the invention provides a method of reducing incidence of a neurodegenerative disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In one embodiment, the invention provides a method of treating a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In another embodiment, the invention provides a method of inhibiting or suppressing a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In one embodiment, the invention provides a method of reducing incidence of a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In another embodiment, the invention provides a method of treating an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In one embodiment, the invention provides a method of inhibiting or suppressing an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In another embodiment, the invention provides a method of reducing incidence of an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

In one embodiment, the invention provides methods for treating neurodegenerative, pulmonary or inflammatory diseases, comprising the step of contacting the subject with a compound represented by formula I and a pharmaceutically acceptable carrier or diluent.

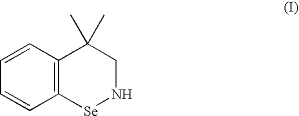

In one embodiment, the glutathione peroxidase mimetic or its isomer, metabolite, and/or salt thereof is represented by the compound of formula (II):

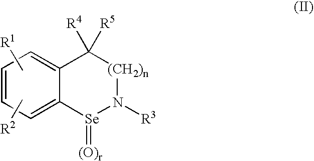

wherein R¹ and R² are independently hydrogen; lower alkyl; OR⁶; —(CH₂)ₘNR⁶R⁷; —(CH₂)_{q}NH₂; —(CH₂)ₘ NHSO₂ (CH₂)₂ NH₂; —NO₂; —CN; —SO₃H; —N⁺(R⁵)₂ O⁻; F; Cl; Br; I; —(CH₂)ₘR⁸; —(CH₂)ₘCOR⁸; —S(O)NR⁶R⁷; —SO₂ NR⁶R⁷; —CO(CH₂)ₚCOR⁸; R⁹;
R³=hydrogen; lower alkyl; aralkyl; substituted aralkyl; —(CH₂)ₘ COR⁸; —(CH₂)_{q}R⁸; —CO(CH₂)ₚ COR⁸; —(CH₂)ₘ SO₂ R⁸; —(CH₂)ₘ S(O)R⁸;
R⁴=lower alkyl; aralkyl; substituted aralkyl; —(CH₂)ₚ COR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae:

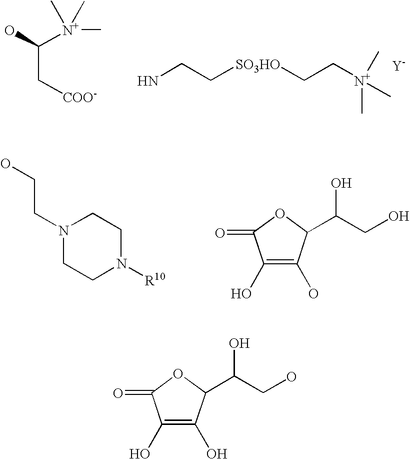

R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

In another embodiment, the glutathione peroxidase mimetic or its isomer, metabolite, and/or salt thereof is represented by the compound of formula (III):

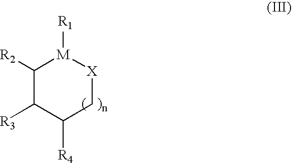

wherein,
   X is O or NH
   M is Se or Te
   n is 0-2
   R¹ is oxygen; and forms an oxo complex with M; or
   R¹ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R⁴ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; wherein R⁴ is not an alkyl; and
wherein if R₂, R₃ and R₄ are hydrogen and R¹ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is O, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 membered ring, substituted by an oxo group a to R₁, and said ring is fused to a second benzene ring, then M is Te;

In another embodiment, the glutathione peroxidase mimetic or its isomer, metabolite, and/or salt thereof is represented by the compound of formula (IV):

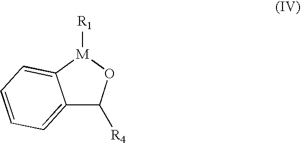

wherein, M, R₁ and R⁴ are as described above.

In another embodiment, the glutathione peroxidase mimetic or its isomer, metabolite, and/or salt thereof is represented by the compound of formula (V):

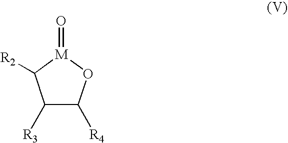

wherein, M, R₂, R₃ and R₄ are as described above;

In another embodiment, the glutathione peroxidase mimetic or its isomer, metabolite, and/or salt thereof is represented by the compound of formula (VI):

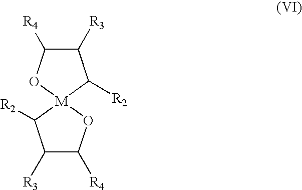

wherein, M, R₂, R₃ and R₄ are as described above;

In another embodiment, the glutathione peroxidase mimetic or its isomer, metabolite, and/or salt thereof is represented by the compound of formula (VII):

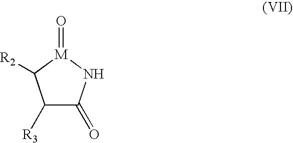

wherein, M, R₂, and R₃ are as described above.

In another embodiment, the glutathione peroxidase mimetic or its isomer, metabolite, and/or salt thereof is represented by the compound of formula (VIII):

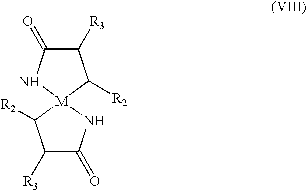

wherein, M, R₂, and R₃ are as described above.

In another embodiment, the glutathione peroxidase mimetic or its isomer, metabolite, and/or salt thereof is represented by the compound of formula (IX)

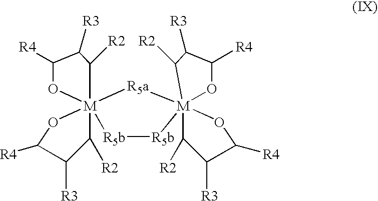

wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R⁴ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

In another embodiment, the glutathione peroxidase mimetic or its isomer, metabolite, and/or salt thereof is represented by the compound of formula (X):

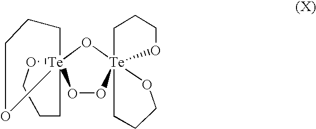

or their combination.

In another embodiment, the glutathione peroxidase mimetic or its isomer, metabolite, and/or salt thereof is represented by the compound of formula (XI):

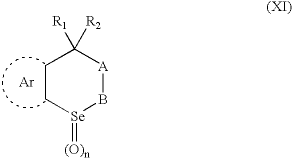

in which: R₁=hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄)ₘ; B=NR₅; O; S; Ar=optionally substituted phenyl or an optionally substituted radical of formula:

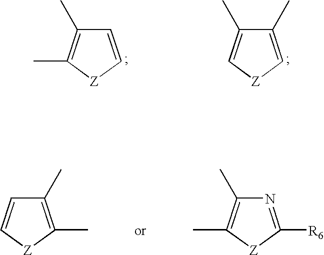

in which: Z=O; S; NR₅; R₃ hydrogen; lower alkyl; optionally substituted aryl; optionally substituted lower aralkyl R₄=hydrogen; lower alkyl; optionally substituted aryl; optionally substituted lower aralkyl; R₅ hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂(aryl); R₆=hydrogen; lower alkyl; optionally substituted aryl; optionally substituted lower aralkyl; optionally substituted heteroaryl; optionally substituted lower heteroaralkyl; trifluoromethyl;

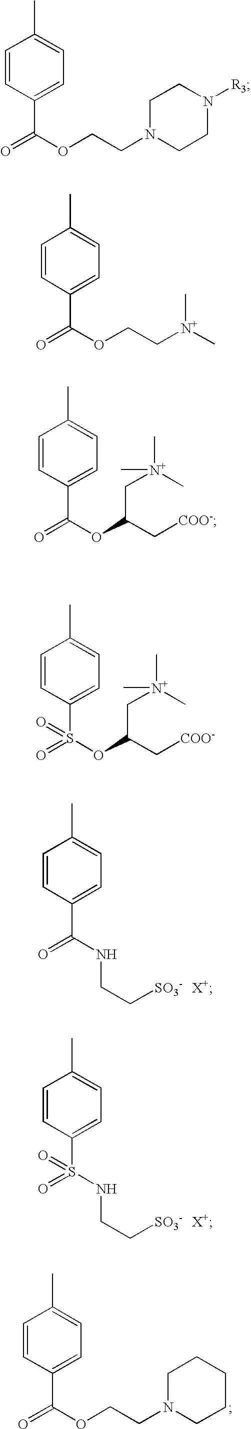

m=0 or 1; n=0 or 1; X⁺ represents the cation of a pharmaceutically acceptable base; and their pharmaceutically acceptable salts of acids or bases.

In other embodiments compounds useful for the purposes herein include 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, and 4,4-dimethyl-thieno-[2,3-e]-isoselenazine-1-oxide.

In another embodiment, the glutathione peroxidase mimetic or its isomer, metabolite, and/or salt thereof is represented by the compound of formula (XII):

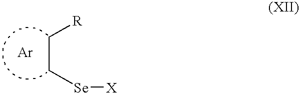

in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N+R₅R₆R₇Y⁻; OR₅; SR₅;
Ar=an optionally substituted phenyl group or an optionally substituted radical of

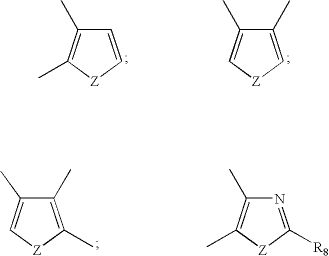

in which Z represents O; S; NR₅; when R=—C(R₁)R₂)-A-B or Ar=a radical of formula

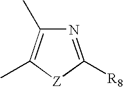

in which Z=O; S; NR₅; when R is hydrogen; X=Ar(R)—Se—; —S-glutathione; —S—N-acetylcysteine; —S-cysteine; —S-penicillamine; —S-albumin; —S-glucose;

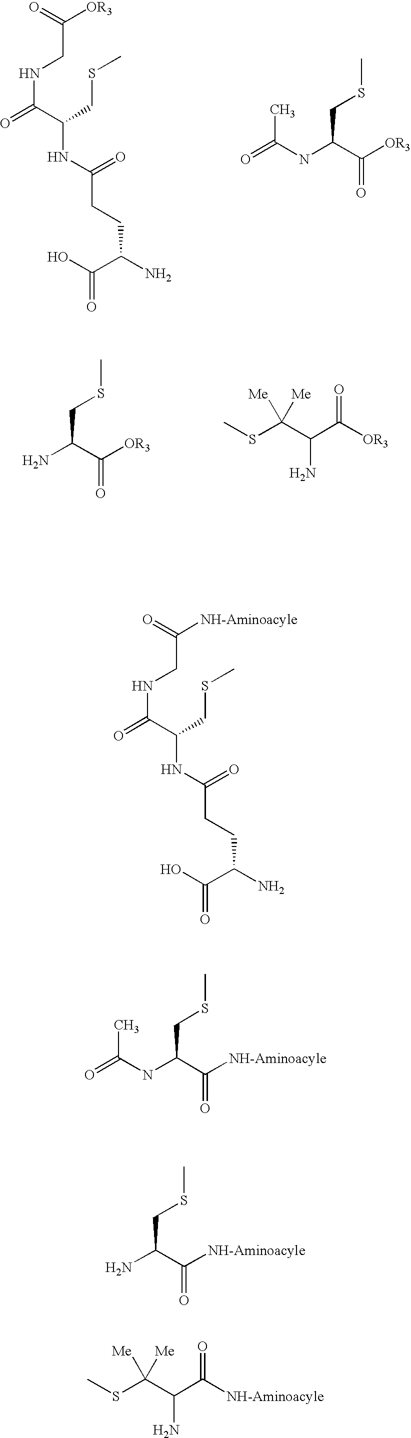

R₃=hydrogen; lower alkyl; optionally substituted aryl, optionally substituted lower aralkyl;
R₄=hydrogen; lower alkyl; optionally substituted aryl: optionally substituted lower aralkyl;
R₅ hydrogen; lower alkyl; optionally substituted aryl: optionally substituted lower aralkyl; optionally substituted heteroaryl; optionally substituted lower heteroaralkyl; CO(lower alkyl); CO(aryl); SO₂(lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl: optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl;

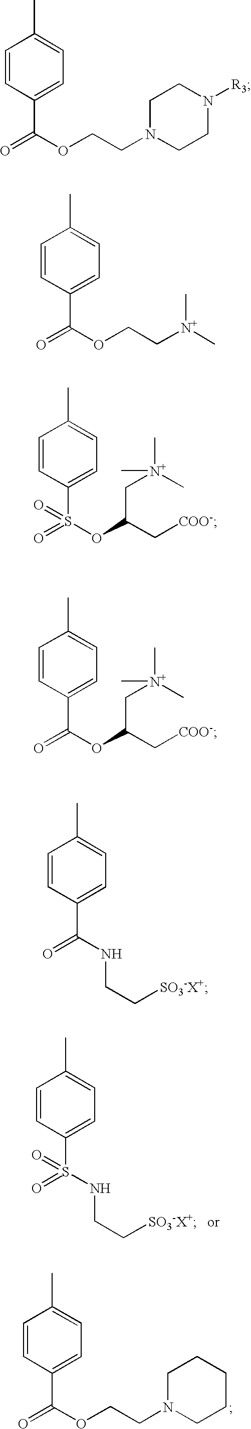

n=0 or 1; X⁺ represents the cation of a pharmaceutically acceptable base;
Y⁻ represents the anion of a pharmaceutically acceptable acid;
and their salts of pharmaceutically acceptable acids or bases.

In one embodiment, provided herein is a method of treating a neurodegenerative disease in a subject, comprising the step of contacting the subject with a glutathione peroxidase mimetic or its isomer, metabolite, and/or salt therefore and one or more of a carbidopa/levodopa, a selegeline, vitamin E, an amantadine, a pramipexole, a ropinerole, coenzyme QIO, a GDNF, an aldosterone inhibitor. an ACE inhibitor, a probucol analog, tacrine, a heptylphysostigmine, simvastatin, lovastatin, pravastatin, thorvastatin, donepezil, or a combination thereof.

In one embodiment, provided herein is a method of treating a pulmonary disease in a subject, comprising the step of contacting the subject with a glutathione peroxidase mimetic or its isomer, metabolite, and/or salt therefore and one or more of a glucocorticoid, a β-2 adrenergic agonist, salmeterol, an anti-cholinergic drug, theophylline, a corticosteroid, a mucolytic agent, an antibiotic, an antiviral, a leukotriene inhibitor or a combination thereof.

In one embodiment, provided herein is a method of treating an inflammatory disease in a subject, comprising the step of contacting the subject with a glutathione peroxidase mimetic or its isomer, metabolite, and/or salt therefore and one or more of a tetracycline compound, a nonsteroidal anti-inflammatory drug, a Cox-2 inhibitor, a corticosteroid, S-adenylmethionine, a synovial fluid supplement, a cetyl myristoleate compound, or a combination thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

This invention relates in one embodiment to compositions and methods for the treatment of neurodegenerative, pulmonary and inflammatory diseases with glutathione peroxidase and its mimetics.

In one embodiment, the term “neurodegenerative disease” refers to disorders characterized by selective neuronal death and the accumulation of insoluble proteinaceous deposits, such as senile plaques and neurofibrillary tangles in Alzheimer's disease (AD), Lewy bodies in Parkinson's disease (PD), and hyaline- and skein-like inclusion bodies in ALS. In another embodiment, oxidative stress plays a critical role in the pathogenesis of progressive neurodegenerative diseases. In one embodiment, both AD and PD have been associated with increased production of reactive oxygen species, resulting in another embodiment from genetic predisposition or environmental factors, such as exposure to pesticides in other embodiments. In one embodiment, the overall levels of oxidative damage to proteins, lipids, and DNA are elevated in AD and PD brains. In another embodiment patients with dementia of the Alzheimer type (DAT) and vascular dementia (VD) show a significant correlation to the antioxidant variables measured in blood samples taken from these patients, demonstrating that VD and DAT diseases are accompanied by oxidative disorders. In one embodiment, the marker for oxidative damage to proteins is the presence of carbonyl groups, which can be introduced into proteins by direct oxidation of Pro, Arg, Lys, and Thr side chains, or by Michael addition reactions with products of lipid peroxidation or glycooxidation. In one embodiment an imbalance between the body's antioxidative capabilities and the level of ROS is a major component in the progression of neurodegenerative diseases.

In one embodiment, the neurodegenerative disease is Alzheimer's disease. In another embodiment, the neurodegenerative disease is Parkinsons disease. In another embodiment, the neurodegenerative disease is Huntington's disease (HD). In another embodiment, the neurodegenerative disease is amyotrophic lateral sclerosis (ALS). In another embodiment, the neurodegenerative disease is Atriplet repeat disease (ARD). In another embodiment, the neurodegenerative disease is Friedreich's Ataxia. In another embodiment, the neurodegenerative disease is stroke. In another embodiment, the neurodegenerative disease is multi-infarct. In another embodiment, the neurodegenerative disease is dementia. In another embodiment, the neurodegenerative disease is multiple sclerosis. In another embodiment, the neurodegenerative disease is chronic fatigue. In another embodiment, the neurodegenerative disease is a combination thereof.

In one embodiment, “Huntington's disease” refers to a fully penetrant autosomal-dominant inherited neurological disorder caused by expanded CAG repeats in the Huntingtin gene. In another embodiment, deficient energy production and increased free radical production are evident in HD, by increases in cerebral lactate in vivo, decreases in mitochondrial complex II-III activity in postmortem tissue, and increased oxidative damage to DNA.

In another embodiment, “Friedreich's Ataxia” refers to the most common form of autosomal recessive ataxia and is characterized in one embodiment, by degeneration of the large sensory neurons extending into the spinal cord, cardiomyopathy and increased incidence of diabetes. In one embodiment, the disease is caused by severely reduced levels of frataxin owing to a large GAA triplet repeat expansion within the first intron of the frataxin gene, causing inhibition of transcriptional elongation. In one embodiment, iron deposits are evident in cardiac tissue of FRDA patients. In another embodiment, cultured fibroblasts from patients exhibit an increased sensitivity to oxidative stress. In one embodiment, a dysfunctional mitochondrial respiratory chain and elevated levels of mitochondrial iron, as a primary or secondary consequence of frataxin deficiency, generate cell-damaging superoxide and hydroxyl radicals through the Fenton reaction. In another embodiment, elevated levels of oxidative stress markers, such as urine 8-hydroxy-2′-deoxyguanosine and serum malondialdehyde, indicative of DNA damage and lipid peroxidation, respectively are reported in patients. In one embodiment, continuous oxidative damage due to an impaired response to oxidative stress further contributes to mitochondrial deficiency and cell degeneration in FRDA.

In one embodiment “amyotrophic lateral sclerosis” (ALS) or “Lou Gehrig's disease”, refers to a progressive, fatal neurodegenerative disorder causing degeneration of the motor neurons of the cortex, brain stem, and spinal cord. In another embodiment, ALS produces progressive weakness of voluntary muscles and eventual death. The onset of disease is usually in the fourth or fifth decade of life, and most affected individuals succumb within 2 to 5 years of disease onset. ALS occurs in both sporadic and familial forms. Of the 5-10% of all cases that are familial (familial ALS or FALS), 20% carry a mutation of the superoxide dismutase I (SODI) gene that codes the ubiquitously expressed Cu,Zn-SOD enzyme. In another embodiment, increased generation of reactive oxygen species (ROS), whose production exceeds the capacity of cellular mechanisms to remove/inactivate it, is a major cause in the progression of the disease.

Accordingly and in one embodiment, provided herein is a composition for treating a neurodegenerative disease in a subject, comprising a glutathione peroxidase mimetic or its isomer, metabolite, and/or salt therefore and pharmaceutically acceptable carrier or diluent.

In another embodiment, neurodegenerative disease for which the compositions provided herein are used is Parkinsons disease (PD), Alzheimers disease (AD), Huntingtons disease (HD), amyotrophic lateral sclerosis (ALS), Atriplet repeat disease (ARD), Friedreich's Ataxia, stroke, multi-infarct, dementia, multiple sclerosis, chronic fatigue syndrome, or a combination thereof.

In another embodiment, the composition for treating a neurodegenerative disease in a subject, comprising a glutathione peroxidase mimetic or its isomer, metabolite, and/or salt therefore and pharmaceutically acceptable carrier or diluent, further comprise one or more agent that is carbidopa/levodopa. In another embodiment, the additional agent is a selegeline. In another embodiment, the additional agent is vitamin E. In another embodiment, the additional agent is an amantadine. In another embodiment, the additional agent is a pramipexole. In another embodiment, the additional agent is a ropinerole. In another embodiment, the additional agent is coenzyme Q10. In another embodiment, the additional agent is a GDNF. In another embodiment, the additional agent is an aldosterone inhibitor. In another embodiment, the additional agent is an ACE inhibitor. In another embodiment, the additional agent is a probucol analog. In another embodiment, the additional agent is tacrine. In another embodiment, the additional agent is a heptylphysostigmine. In another embodiment, the additional agent is simvastatin. In another embodiment, the additional agent is lovastatin. In another embodiment, the additional agent is pravastatin. In another embodiment, the additional agent is thorvastatin. In another embodiment, the additional agent is donepezil. In another embodiment, the additional agent is a combination thereof.

In one embodiment “pulmonary disease” or “pulmonary disorders”, refer to diseases or disorders affecting the respiratory system, resulting in obstructed breathing, hypoxemia, hypercapnia and lung tissue damage. In one embodiment, obstructive diseases of the airways are characterized by airflow limitation due to constriction of airway smooth muscle, edema and hypersecretion of mucous leading to increased work in breathing, dyspnea, hypoxemia and hypercapnia.

In another embodiment, lung cells, in particular alveolar epithelial type II cells, are susceptible to the injurious effects of oxidants. In one embodiment, lung cells release inflammatory mediators and cytokines/chemokines such as tumour necrosis factor-α (TNF-α), interleukin (IL)-1 and IL-8 in response to oxidative/nitrosative stress. In one embodiment, release of cytokines/chemokines induces neutrophil recruitment and the activation of key transcription factors such as activator protein-1 (AP-1), thereby augmenting the inflammatory response and tissue damage. In another embodiment, the acute and chronic alveolar and/or bronchial inflammatory response is a fundamental process involved in the pathogenesis of many lung diseases such as asthma in one embodiment, or chronic obstructive pulmonary disease (COPD), acute respiratory distress syndrome (ARDS), idiopathic pulmonary fibrosis (IPF) and cystic fibrosis (CF) in other embodiments.

In one embodiment, the pulmonary disease sought to be treated with the compositions provided herein, is chronic obstructive pulmonary disease (COPD). In another embodiment, the pulmonary disease is cystic fibrosis (CF). In another embodiment, the pulmonary disease is dyspnea. In another embodiment, the pulmonary disease is emphysema. In another embodiment, the pulmonary disease is wheezing. In another embodiment, the pulmonary disease is reactive airway disease. In another embodiment, the pulmonary disease is pulmonary hypertension. In another embodiment, the pulmonary disease is pulmonary fibrosis. In another embodiment, the pulmonary disease is hyper-responsive airways. In another embodiment, the pulmonary disease is pulmonary bronchoconstriction. In another embodiment, the pulmonary disease is respiratory tract inflammation or allergies. In another embodiment, the pulmonary disease is chronic bronchitis. In another embodiment, the pulmonary disease is bronchoconstriction. In another embodiment, the pulmonary disease is Acute Respiratory Distress Syndrome (ARDS). In another embodiment, the pulmonary disease is infantile Respiratory Distress Syndrome (infantile RDS). In another embodiment, the pulmonary disease is allergic rhinitis. In another embodiment, the pulmonary disease is lung cancer. In another embodiment, the pulmonary disease is chronic bronchitis In another embodiment, the pulmonary disease is a combination thereof.

In one embodiment, “Acute Respiratory Distress Syndrome” (ARDS), refers to an aggressive inflammatory response accompanied by pulmonary hypertension, which can lead to multiple organ failure and a high rate of mortality ranging from 35% to 50%. In another embodiment, Acute Respiratory Distress Syndrome (ARDS), or stiff lung, shock lung, pump lung and congestive atelectasis, caused by fluid accumulation within the lung which, in turn, causes the lung to stiffen. The condition is triggered within 48 hours by a variety of processes that injure the lungs such as trauma, head injury, shock, sepsis, multiple blood transfusions, medications, pulmonary embolism, severe pneumonia, smoke inhalation, radiation, high altitude, near drowning, and others. In one embodiment, ARDS occurs as a medical emergency and may be caused by other conditions that directly or indirectly cause the blood vessels to “leak” fluid into the lungs. In ARDS, the ability of the lungs to expand is severely decreased and produces extensive damage to the air sacs and lining or endothelium of the lung.

In another embodiment, the inflammatory response is the result of inflammatory cell migration into interstitial and alveolar spaces followed by release of proteases and reactive oxygen intermediates.

In another embodiment, the term “chronic obstructive pulmonary disease (COPD)” encompasses chronic obstructive bronchitis, with obstruction of small airways, and emphysema, with enlargement of air spaces and destruction of lung parenchyma, loss of lung elasticity, and closure of small airways and refers to a pulmonary disorder characterized by breathlessness, cough and sputum, with chronic airway obstruction and lung hyperinflation as a result of chronic bronchitis and emphysema (dilation of the distal lung airspaces). In another embodiment, chronic bronchial hypereactivity which is prominent in bronchial asthma is also found in COPD. Airway remodelling in COPD leads in one embodiment to persistent and irreversible airway narrowing and mucus hypersecretion. In another embodiment, abnormalities in the airway smooth muscle function results in decreased or impaired relaxation or increased contractility of the airways, contributing to COPD. In one embodiment, inhaled ROS or in another embodiment, ROS released from activated neutrophils, causes the inactivation of α₁-proteinase inhibitor (α₁-PI), producing a functional deficiency of α₁-PI in the airspaces, an event that is critical in another embodiment to the proteinase/antiproteinase imbalance. In one embodiment, an increase in the concentration of hydrogen peroxide is evident in the exhaled breath condensates of patients with COPD, particularly during exacerbations. In another embodiment, iron is normally bound to various iron-binding compounds, such as transferrin, ceruloplasmin, and ferritin. In another embodiment, this protective mechanism is disrupted in the lungs of COPD patients, since oxidants release in another embodiment, the iron from ferritin.

In one embodiment, “cystic fibrosis” is caused by a mutation in the CFTR gene, and refers to the spontaneous mucus accumulation and goblet cell metaplasia, leading in one embodiment, to the formation of mucus plugs and plaques, which in turn results in airway obstruction. In another embodiment, a defective or missing CFTR results in crippling GSH transport, resulting in a deficiency of extracellular GSH and supraphysiological levels of intracellular GSH, decreasing the lungs endogenous capacity to remove ROS.

Accordingly and in one embodiment, provided herein is a composition for treating a pulmonary disease in a subject, comprising a glutathione peroxidase mimetic or its isomer, metabolite, and/or salt therefore and pharmaceutically acceptable carrier or diluent.

In another embodiment, the pulmonary disease sought to be treated using the compositions and methods provided herein, is chronic obstructive pulmonary disease (COPD). In another embodiment, the pulmonary disease is cystic fibrosis (CF). In another embodiment, the pulmonary disease is dyspnea. In another embodiment, the pulmonary disease is emphysema. In another embodiment, the pulmonary disease is wheezing. In another embodiment, the pulmonary disease is pulmonary hypertension. In another embodiment, the pulmonary disease is pulmonary fibrosis. In another embodiment, the pulmonary disease is hyper-responsive airways. In another embodiment, the pulmonary disease is pulmonary bronchoconstriction. In another embodiment, the pulmonary disease is respiratory tract inflammation or allergies. In another embodiment, the pulmonary disease is chronic bronchitis. In another embodiment, the pulmonary disease is bronchoconstriction. In another embodiment, the pulmonary disease is Acute Respiratory Distress Syndrome (ARDS). In another embodiment, the pulmonary disease is infantile Respiratory Distress Syndrome (infantile RDS). In another embodiment, the pulmonary disease is allergic rhinitis. In another embodiment, the pulmonary disease is lung cancer. In another embodiment, the pulmonary disease is chronic bronchitis. In another embodiment, the pulmonary disease is a combination thereof.

In one embodiment, provided herein is a composition for treating a pulmonary disease comprising a glutathione peroxidase mimetic or its isomer, metabolite, and/or salt therefore and one or more additional agent that is a glucocorticoid. In another embodiment, the one or more additional agent is a β-2 adrenergic agonist. In another embodiment, the one or more additional agent is salmeterol. In another embodiment, the one or more additional agent is an anti-cholinergic drug. In another embodiment, the one or more additional agent is Theophylline. In another embodiment, the one or more additional agent is a corticosteroid. In another embodiment, the one or more additional agent is a mucolytic agent. In another embodiment, the one or more additional agent is an antibiotic. In another embodiment, the one or more additional agent is an antiviral. In another embodiment, the one or more additional agent is a leukotriene inhibitor. In another embodiment, the one or more additional agent is a combination thereof.

In one embodiment “inflammatory dsease” refers to a disease process associated with activation of leukocytes for more than six (6) months. In another embodiment, which leads in other embodiments, to damaged organs, tissues or both. In one embodiment, reactive oxygen species produced in one embodiment by activated phagocytes are involved in inflammatory processes and contribute in another embodiment, to cell and tissue damage either directly or through activation of proteases. In one embodiment, an inverse correlation is found between antioxidant levels and inflammation In one embodiment, the inflammatory disease treated using the methods and compositions provided herein, is rheumatoid arthritis. In another embodiment, the inflammatory disease is Behcet's disease. In another embodiment, the inflammatory disease is polyarteritis nodosa. In another embodiment, the inflammatory disease is Wegener granulomatosis. In another embodiment, the inflammatory disease is Takaysu's arteritis. In another embodiment, the inflammatory disease is osteoarthritis. In another embodiment, the inflammatory disease is lupus. Juvenile idiopathic arthritis. In another embodiment, the inflammatory disease is alcoholic cirrhosis. In another embodiment, the inflammatory disease is a combination thereof.

In one embodiment, “rheumatoid arthritis” (RA) refers to a disorder wherein synovial hyperplasia and inflammatory cell recruitment occurs, and, in its later stages, cartilage a bone destruction. The presence of a large number of activated T cells in the synovial membrane is indicates that in another embodiment RA is an immune-mediated disease.

In one embodiment, an inverse correlation exists between dietary intake of antioxidants and RA incidence, and in another embodiment, an inverse correlation exists between antioxidant levels and inflammation levels. In another embodiment iron, a catalyst for hydroxyl radical production from hydrogen peroxide is present in RA synovial tissue and is associated in another embodiment, with poorer RA prognosis. In one embodiment RA sera and synovial fluids show increased oxidative enzyme activity along with decreased antioxidant levels. In one embodiment RA synovial fluid and tissue have demonstrated oxidative damage to hyaluronic acid, or lipid peroxidation products, oxidized low-density-lipid proteins (LDL), and increased carbonyl groups in other embodiments, reflective of oxidation damage to proteins. In another embodiment, oxidative damage in RA patients to cartilage, extracellular collagen, and intracellular DNA has also been demonstrated is evident. In one embodiment, oxidative stress induces T cell hyporesponsiveness in RA through effects on proteins and proteosomal degradation.

In one embodiment, “Wegener granulomatosis”, or “granulomatous vasculitis”, refers to a disease that produces inflammation of the medium and small arteries and venules. In another embodiment, necrotizing and crescentic changes are found in the glomeruli. In one embodiment, the process affects the upper and lower airways and kidneys. In one embodiment, Wegener granulomatosis (WG) is characterized by systemic necrotizing vasculitis and crescentic glomerulonephritis shows no vascular immunoglobulin depositionin another embodiment, anti-neutrophil cytoplasmic autoantibodies (ANCA) are in the serum of −80% of patients with pauci-immune necrotizing vasculitis such as WG and polyarteritis nodosa (PN). The vascular lesions form a continuum from renal-limited necrotizing and crescentic glomerulonephritis to systemic vasculitis. In one embodiment, ANCA react with constituents of neutrophil primary granules and monocyte lysosomes. In one embodiment, ANCA are capable of activating neutrophils and cause the release of reactive oxygen species (ROS) and primary granule contents from ANCA-stimulated human neutrophils. In one embodiment, the same abovementioned mechanism exists in Polyarteritis nodosa.

In one embodiment “Beh [symbol] et's disease” (BD) refers to a systemic form of primary vasculitis characterized by recurrent oral and genital ulcers and ocular inflammation and with frequent involvement of the joints, central nervous system and gastrointestinal tract. In one embodiment, glutathione peroxidase (GPX) activity is lower in BD patients. In another embodiment, MDA, a marker of oxidative stress on proteins, is markedly higher in BD patients.

In one embodiment, “Polyarteritis nodosa” refers to an autoimmune disease characterized by spontaneous inflammation of the arteries (arteritis) of the body. In another embodiment PN may affect any organ of the body, such as in certain embodiment, muscles, joints, intestines, nerves, kidneys, and skin.

Accordingly and in one embodiment, provided herein is a composition for treating an inflammatory disease in a subject, comprising a glutathione peroxidase mimetic or its isomer, metabolite, and/or salt therefore and pharmaceutically acceptable carrier or diluent.

In one embodiment, the inflammatory sought to be treated using the compositions and methods provided herein is rheumatoid arthritis, Behcet's disease, polyarteritis nodosa, Wegener granulomatosis, lupus, juvenile idiopathic arthritis, alcoholic cirrhosis or a combination thereof.

In another embodiment, the compositions for treating an inflammatory diseases provided herein, comprise a glutathione peroxidase mimetic or its isomer, metabolite, and one or more agent, that is a tetracycline compound. In another embodiment, the additional agent is a nonsteroidal anti-inflammatory drug. In another embodiment, the additional agent is a Cox-2 inhibitor. In another embodiment, the additional agent is a corticosteroid. In another embodiment, the additional agent is S-adenylmethionine. In another embodiment, the additional agent is a synovial fluid supplement. In another embodiment, the additional agent is a cetyl myristoleate compound. In another embodiment, the additional agent is a combination thereof.

In one embodiment, superoxide anion (O⁻₂) formation from oxygen is the first step. O⁻₂ is generated primarily by mitochondrial metabolism, molybdenum hydroxylase (xanthine, sulfite, and aldehyde oxidases) reactions, arachidonic acid metabolism, and NADPH oxidase-dependent processes in phagocytic cells. Reaction of O⁻₂ and hydrogen peroxide (H₂O₂) in the presence of transition metal, usually ferrous iron (Fe⁺⁺), produces the hydroxyl radical (.⁻OH). When catalyzed by neutrophil myeloperoxidase (MPO), H₂O₂ and a chloride form hypochlorous acid (HOCl). .⁻OH and HOCl are emphasized because both are extremely potent oxidants. H₂O₂ gains significance as a central precursor to both ⁻OH and HOCl

Oxidative Stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, overproduction of reactive oxygen species (ROS) including hydrogen peroxide (H₂O₂), superoxide anion (.O⁻₂⁻); nitric oxide (.NO) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of .O⁻₂⁻ and H₂O₂. First, SOD catalyses the dismutation of .O₂⁻ to H₂O₂ and molecular oxygen (O₂), resulting in selective .O₂⁻ scavenging. Then, GPX and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When in another embodiment, free radicals are generated in excess or when FRSEs are defective, H₂O₂ is reduced into hydroxyl radical (.OH), which is one of the highly reactive ROS responsible in one embodiment for initiation of lipid peroxidation of cellular membranes. In another embodiment, organic peroxide-induced lipid peroxidation is implicated as one of the essential mechanisms of toxicity in keratinocytes. In one embodiment, benzoyl peroxide, a topical agent, shows the ability to induce an inflammatory reaction mediated by oxidative stress in addition to its antibacterial activity, thereby increasing lipid peroxidation. In one embodiment, an indicator of the oxidative stress in the cell is the level of lipid peroxidation and its final product is MDA. In another embodiment the level of lipid peroxidation increases in inflammatory diseases. In one embodiment, the compounds provided herein and in another embodiment, are represented by the compounds of formula I-XII, are effective antioxidants, capable of reducing lipid peroxidation, or in another embodiment, are effective as anti-inflammatory agents.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

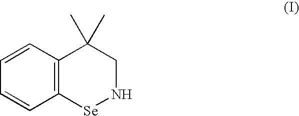

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet.

In one embodiment, oxidized low-density lipoprotein (oxLDL) interacts with β₂-glycoprotein-I, forming oxLDL/β₂GPI complexes. In another embodiment, autoimmune vascular inflammation or oxidative stress in another embodiment promote the formation of these complexes. In one embodiment, patients with systemic lupus erythematosus (SLE) and systemic sclerosis (SSc), the high serum levels and prevalence of circulating oxLDL/β₂GPI complexes and IgG anti-oxLDL/β₂GPI antibodies indicate significant vascular oxidative stress in one embodiment. In one embodiment, the compounds provided herein, are effective in inhibiting or suppressing the formation of oxLDL/β₂GPI complexes, thereby reducing antibodies specific against oxLDL/β₂GPI complexes, acting as an anti-atherogenic agent.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues must be able to protect themselves against the toxic effects of ROS. In one embodiment ROS-detoxifying enzymes have an important role in cutaneous wound repair.

In one embodiment, the glutathione (GSH) system controls cellular redox states and in another embodiment, it is a primary defense mechanism for H₂O₂ and peroxide removal in brain. In another embodiment, GSHPx is localized in both brain astrocytes and neurons.

In another embodiment, glutathione peroxidase, is important in the body's defense mechanism against oxygen burst. In vitro and in vivo studies with the compound of formula I, show in one embodiment, that glutahion peroxidase is capable of protecting cells against reactive oxygen species.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx or a mimetic thereof, or its pharmaceutically acceptable salt, its functional derivative, its synthetic analog or a combination thereof, is used in the methods and compositions of the invention.

In one embodiment, the glutathione peroxidase mimetic is represented by formula I

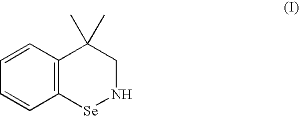

In one embodiment, the compound of formula (II), refers to benzisoselen-azoline or -azine derivatives represented by the following general formula:

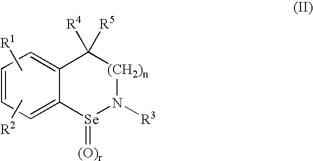

where: R¹, R²=hydrogen; lower alkyl; OR⁶; —(CH₂)ₘ NR⁶R⁷; —(CH₂)_{q}NH₂; —(CH₂)ₘ NHSO₂ (CH₂)₂ NH₂; —NO₂; —CN; —SO₃H; —N⁺(R⁵)₂ O⁻; F; Cl; Br; I; —(CH₂)ₘ R⁸; —(CH₂)ₘ COR⁸; —S(O)NR⁶R⁷; —SO₂ NR⁶R⁷; —CO(CH₂)ₚ COR⁸; R⁹; R³=hydrogen; lower alkyl; aralkyl; substituted aralkyl; —(CH₂)ₘ COR⁸; —(CH₂)_{q}R⁸; —CO(CH₂)ₚ COR⁸; —(CH₂)ₘ SO₂ R⁸; —(CH₂)ₘ S(O)R⁸; R⁴=lower alkyl; aralkyl; substituted aralkyl; —(CH₂)ₚ COR⁸; —(CH₂)ₚR⁸; F; R⁵=lower alkyl; aralkyl; substituted aralkyl; R⁶=lower alkyl; aralkyl; substituted aralkyl; —(CH₂)ₘCOR⁸; —(CH₂)_{q}R⁸; R⁷=lower alkyl; aralkyl; substituted aralkyl; —(CH₂)ₘCOR⁸; R⁸=lower alkyl; aralkyl; substituted aralkyl; aryl; substituted aryl; heteroaryl; substituted heteroaryl; hydroxy; lower alkoxy; R⁹; R⁹=

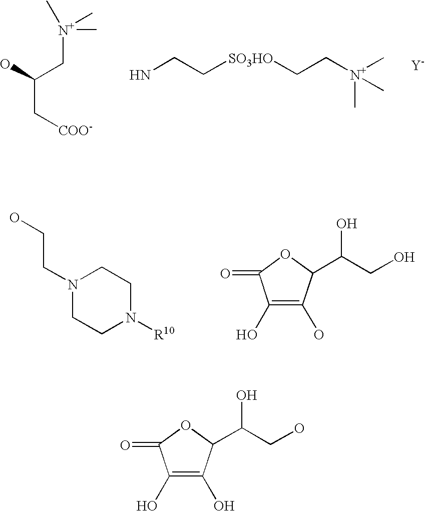

R¹⁰=hydrogen; lower alkyl; aralkyl or substituted aralkyl; aryl or substituted aryl; Y represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like. In one embodiment, the glutathione peroxidase mimetic or its isomer, metabolite, and/or salt therefore, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating neurodegenerative, pulmonary and inflammatory diseases with organoselenium compounds, thereby increasing endogenous anti-oxidant ability of the skin, or in another embodiment, scavenging free radicals causing the symptoms associated in certain embodiments with the neurodegenerative, pulmonary and inflammatory diseases described herein.

In one embodiment, the glutathione peroxidase mimetic compounds provided herein are effective in inhibiting or suppressing inflammatory processes. In another embodiment, the glutathione peroxidase mimetic compounds provided herein are effective in reducing incidence of inflammatory processes. In one embodiment, the glutathione peroxidase mimetic compounds provided herein are effective in removing reactive oxygen species from contacted cells or organs or tissues.

In one embodiment compounds capable of scavanging free oxygen radicals, or in another embodiment, compounds capable of increasing endogenous anti-oxidant abilities are helpful in the treatment of neurodegenerative, pulmonary and inflammatory diseases described herein. In another embodiment the compounds capable of scavanging free oxygen radicals, or in another embodiment, compounds capable of increasing endogenous anti-oxidant abilities are the glutathione peroxidase mimetics described in the compositions provided herein.

In another embodiment, the glutathione peroxidase mimetic or its isomer, metabolite, and/or salt therefore used in the compositions and methods provided herein, is represented by the compound of formula III:

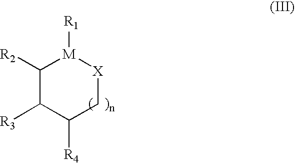

wherein,
the compound of formula I is a ring; and
   X is O or NH
   M is Se or Te
   n is 0-2
   R¹ is oxygen; and
   forms an oxo complex with M; or
   R₁ is oxygen or NH; and
forms together with the metal, a 4-7 member ring, which optionally is substituted by an oxo group; or
forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
R₂, R₃ and R₄ are independently hydrogen, alkyl, oxo, amino or together with the organometalic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl;
wherein R⁴ is not an alkyl; and
wherein if R₂, R₃ and R₄ are hydrogen and R¹ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometalic ring a fused benzene ring, R₄ is hydrogen, then M is Se; or
if R₄ is an oxo group, and R₂ and R₃ form together with the organometalic ring a fused benzene ring, R₁ is oxygen, n is 0 and forms together with the metal a first 5 member ring, substituted by an oxo group a to R₁, and said ring is fused to a second benzene ring, then M is Te.

In one embodiment, a 4-7 member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7 member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, is —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In another embodiment, a compound of formula IV is provided, wherein M, R₁ and R₄ are as described above.

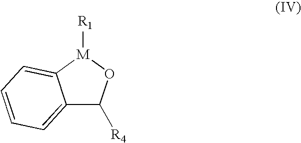

In another embodiment, a compound of formula V is provided, wherein M, R₂, R₃ and R₄ are as described above.

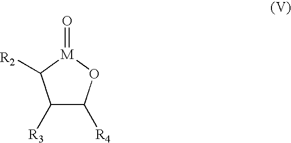

In another embodiment, a compound of formula VI is provided, wherein M, R₂, R₃ and R₄ are as described above.

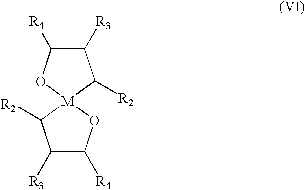

In another embodiment, a compound of formula (VII) is provided, wherein M, R₂ and R₃ are as described above.

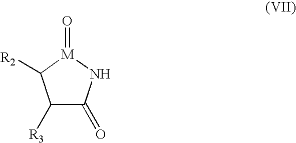

In another embodiment, a compound of formula VIII is provided, wherein M, R₂ and R₃ are as described above.

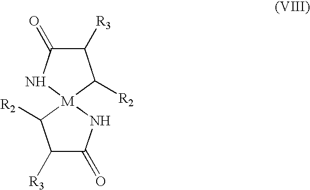

In one embodiment, the compound of formula III, used in the compositions and methods provided herein, is represented by any one of the following compounds or their combinations:

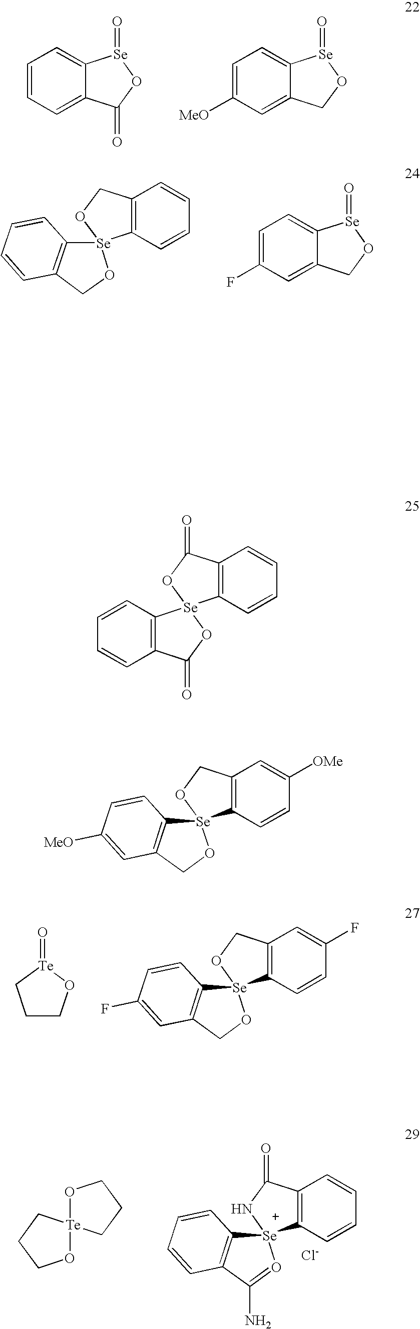

In another embodiment, the glutathione peroxidase mimetic or its isomer, metabolite, and/or salt therefore used in the compositions and methods provided herein, is represented by the compound of formula IX:

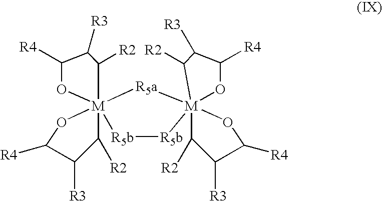

wherein,
M is Se or Te;

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R⁴ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

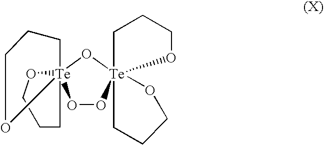

In another embodiment, the glutathione peroxidase mimetic or its isomer, metabolite, and/or salt thereof is represented by the compound of formula (XI):

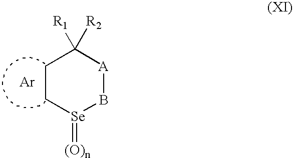

in which: R₁=hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄); B=NR₅; O; S; Ar=optionally substituted phenyl or an optionally substituted radical of formula:

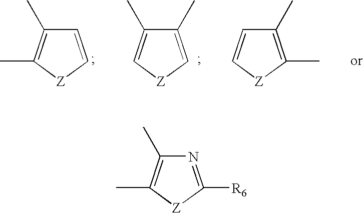

in which: Z=O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂(aryl); R₆=hydrogen; lower alkyl; optionally substituted aryl; optionally substituted lower aralkyl; optionally substituted heteroaryl; optionally substituted lower heteroaralkyl; trifluoromethyl;

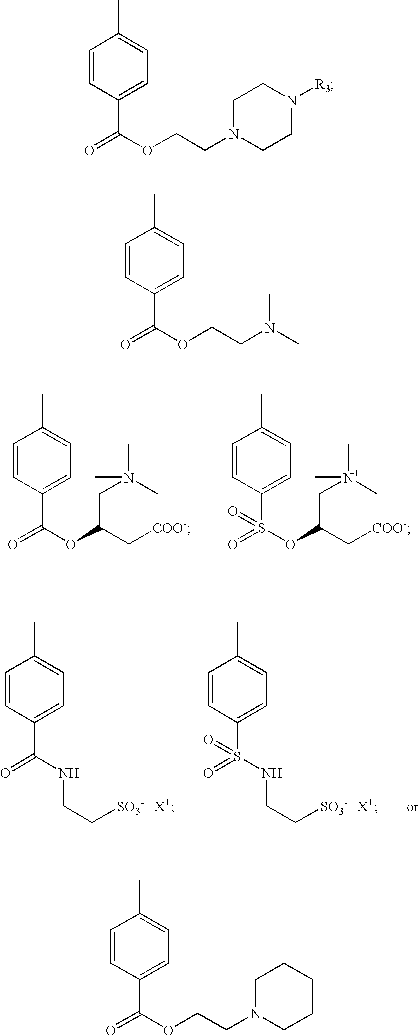

m=0 or 1; n=0 or 1; X⁺ represents the cation of a pharmaceutically acceptable base; and their pharmaceutically acceptable salts of acids or bases. In some embodiments, when B=NR₅ with R₅ is hydrogen, lower alkyl, optionally substituted lower aralkyl, CO(lower alkyl), and A=CO or (—CH₂—)ₘ, then Ar is different from an optionally substituted phenyl.

In other embodiments compounds usefule for the purposes herein include 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, and 4,4-dimethyl-thieno-[2,3-e]-isoselenazine-1-oxide.

In another embodiment, the glutathione peroxidase mimetic or its isomer, metabolite, and/or salt thereof is represented by the compound of formula (XII):

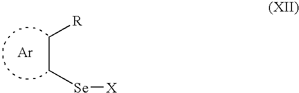

in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N⁺R₅R₆R₇Y⁻; OR₅; SR₅;
Ar=an optionally substituted phenyl group or an optionally substituted radical of

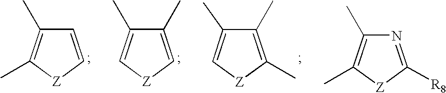

in which Z represents O; S; NR₅; when R=—C(R₁R₂)-A-B or Ar=a radical of formula

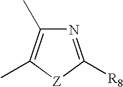

in which Z=O; S; NR₅; when R is hydrogen; X=Ar(R)—Se—; —S-glutathione; —S—N-acetylcysteine; —S-cysteine; —S-penicillamine; —S-albumin; —S-glucose;

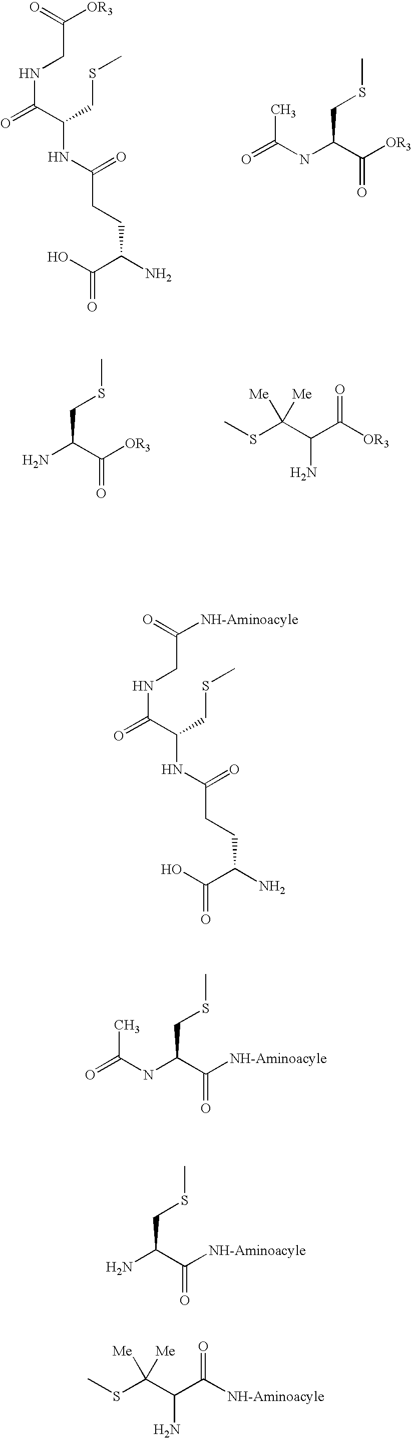

R₃=hydrogen; lower alkyl; optionally substituted aryl, optionally substituted lower aralkyl;
R₄=hydrogen; lower alkyl; optionally substituted aryl: optionally substituted lower aralkyl;
R₅=hydrogen; lower alkyl; optionally substituted aryl: optionally substituted lower aralkyl; optionally substituted heteroaryl; optionally substituted lower heteroaralkyl; CO(lower alkyl); CO(aryl); SO₂(lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl: optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl;

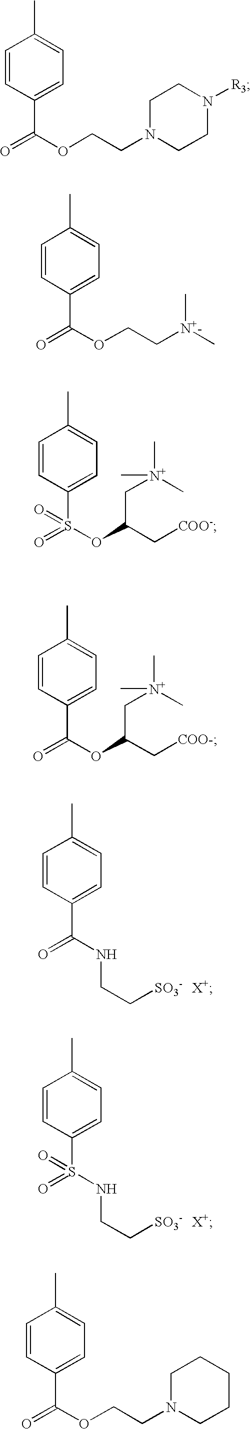

n=0 or 1; X⁺ represents the cation of a pharmaceutically acceptable base;
Y⁻ represents the anion of a pharmaceutically acceptable acid;
and their salts of pharmaceutically acceptable acids or bases.

In other embodiments, organoselenium compounds of formula (XII), include di[2-[2′-(1′-amino-2′-methyl)propyl]phenyl]-diselenide; di[2-[2′-(1′-amino-2′-methyl)propyl]phenyl]-diselenide dihydrochloride; di[2-[2′-(1′-ammonium-2′-methyl)propyl]phenyl]-diselenide di-paratoluenesulphonate; di[2-[2′-(1′-amino-2′-methyl)propyl]-4-methoxy]phenyl-diselenide; di[2-[2′-(1′-methylamino-2′-methyl)propyl]phenyl]-diselenide; di[2-[2′-(1′-methylamino-2′-methyl)propyl]phenyl]-diselenide dihydrochloride; di[2-[2′-(1′-dimethylamino-2′-methyl)propyl]phenyl]-diselenide; di[2-[2′-(1′-trimethylammonium-2′-methyl)propyl]phenyl]-diselenide di-paratoluenesulphonate; S—(N-acetyl-L-cysteinyl)-[2-[2′-(1′-amino-2′-methyl)-propyl]phenyl]-selenide; and S-glutathionyl-[2-[2′-(1-amino-2′-methyl)-propyl]-phenyl]-selenide.

In one embodiment, the compounds represented by formula I-XII, mimic the in-vivo activity of glutathione peroxidase. The term “mimic” refers, in one embodiment to comparable, identical, or superior activity, in the context of conversion, timing, stability or overall performance of the compound, or any combination thereof.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the compositions provided herein are used for the treatment of neurodegenerative, pulmonary and inflammatory diseases described herein and may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In some embodiments, the compositions and methods provided herein permit direct application to the site where it is needed. In the practice of the methods provided herein, it is contemplated that virtually any of the compositions provided herein can be employed.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein are suitable for oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical, or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

In one embodiment, the method of the invention comprises administering a the compositions provided herein via an intradermal patch. The method in some embodiments also comprises administering the patch adjacent to the area of skin to be treated. As used herein a “patch” comprises at least the compositions provided herein and a covering layer, such that, the patch can be placed over the area of skin to be treated. In another embodiment, the patch is designed to maximize delivery of the compositions provided herein through the stratum corneum and into the epidermis or dermis, reduce lag time, promote uniform absorption, and reduce mechanical rub-off.

In some embodiments, the method comprises administering a topical formulation of the compositions provided herein to an affected site of skin. In some embodiments, topical administration according to the present invention comprises aerosol, cream, foam, gel, liquid, ointment, paste, powder, shampoo, spray, patch, disk, or dressing.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I-XII are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration.

In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In one embodiment, liposomes (vesicles) are completely closed lipid bilayer membranes containing an entrapped aqueous volume. Liposomes in one embodiment may be unilamellar vesicles (possessing a single membrane bilayer) or multilamellar vesicles (onion-like structures characterized by multiple membrane bilayers, each separated from the next by an aqueous layer). Liposomes smaller than about 200 nm usually only consist of one bilayer (unilamellar liposomes) but larger liposomes can contain concentric layers of lipid or several smaller liposomes can be formed inside large liposomes. These larger multicompartmented liposomes are known as multilamellar liposomes. Multivesicular liposomes are liposomes containing multiple non-concentric chambers within each liposome particle, resembling a “foam-like” matrix.

The original liposome preparation of Bangham et al. (J. Mol. Biol., 1965, 12 pp. 238-252) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. MLVs so formed may be used in the practice of the present invention.

Another class of multilamellar liposomes embodied herein are characterized as having substantially equal lamellar solute distribution. This class of liposomes is denominated as stable plurilamellar vesicles (SPLV) as defined in U.S. Pat. No. 4,522,803 to Lehk, et al., reverse phase evaporation vesicles (REV) as described in U.S. Pat. No. 4,235,871 to Papahadjopoulos et al., monophasic vesicles as described in U.S. Pat. No. 4,558,579 to Fountain, et al., and frozen and thawed multilamellar vesicles (FATMLV) wherein the vesicles are exposed to at least one freeze and thaw cycle; this procedure is described in Bally et al., PCT Publication No. 87/00043, Jan. 15, 1987, entitled “Multilamellar Liposomes Having Improved Trapping Efficiencies”; these references are incorporated herein by reference.

Liposomes are comprised of lipids; the term lipid as used herein shall mean any suitable material resulting in a bilayer such that a hydrophobic portion of the lipid material orients toward the interior of the bilayer while a hydrophilic portion orients toward the aqueous phase. Exemplary but non-limiting lipids which can be used in the liposome formulations of the present invention are the phospholipids such as phosphatidylcholine (PC) and phosphatidylglycerol (PG), more particularly dimyristoylphosphatidylcholine (DMPC) and dimyristoylphosphatidylglycerol (DMPG). Liposomes may be formed and vesiculated using DMPG, or DMPG mixed with DMPC in, for example, a 3:7 mole ratio, respectively.

During preparation of the liposomes, organic solvents may be used to suspend the lipids. Suitable organic solvents are those with intermediate polarities and dielectric properties, which solubilize the lipids, and include but are not limited to halogenated, aliphatic, cycloaliphatic, or aromatic-aliphatic hydrocarbons, such as benzene, chloroform, methylene chloride, or alcohols, such as methanol, ethanol, and solvent mixtures such as benzene:methanol (70:30). As a result, solutions (mixtures in which the lipids and other components are uniformly distributed throughout) containing the lipids are formed. Solvents are generally chosen on the basis of their biocompatability, low toxicity, and solubilization abilities.

In further embodiments of liposome formulations of the compounds and compositions embodied here for the purposes hereof, such liposomes include soft vesicles, that are formed in a hydroalcoholic medium containing C₂-C₄ alcohols. Such liposomes when applied to the skin, change their size by fusing together as a result of the change in solvent ratio. In the formulation, the vesicle size does not change since the ratio between the solvents is constant. Penetration and evaporation of the alcohol following application to the skin allows the transition from small to large vesicles, which grow in size until a film is formed. In such embodiments, liposomes comprise vesicles in a size range up to about 1 micrometer, but can range from nanometers to millimeters. Such compositions comprise from 0.5% to 10% phospholipids, from 20% to 50% of ethanol, from 0 to 20% propylene glycol, at least 20% water, and the glutathione peroxidase mimetic or combination thereof, where the combined ethanol and propylene glycol content does not exceed 70%. In one embodiment, the glutathione peroxidase mimetic is the compound of formula (I). In other embodiments, compounds of formulas (II)-(XII) are contained therein. In other embodiments, such liposomes comprise 22 to 70% of a combination of the ethanol and propylene glycol, and more than 20% water. In still other embodiments, the amount of ethanol is between 20 and 50 weight-% of the composition, the content of water being at least about 25 weight-%. In yet other embodiment, a short chain C₃-C₄ alcohol can be used in addition to ethanol, such as but not limited to propanol, isopropanol, butanol, iso-butanol and t-butanol. In non-limiting examples of such liposomes, the composition can comprise 0.5% to 10% phospholipids, from 5% to 35% of a C₃ or C₄ alcohol, 15 to 30% ethanol, wherein the combined content of ethanol and C₃ or C₄ alcohol is at least 20 wt. % and not more than 40 wt. %, up to 20 wt. % glycol, such as propylene glycol, at least 20% water, encapsulating one or a combination of glutathione peroxidase mimetics described herein. In one embodiment, the glutathione peroxidase mimetic is the compound of formula (I). In other embodiments, compounds of formulas (II)-(XII) are contained therein. In another embodiment, the liposomal composition comprises from 0.5% to 10% phospholipids, from 5% to 35% of a C₃ or C₄ alcohol, 15% to 30% ethanol, where the C₃ or C₄ alcohol is at least 20 wt. % and not more than 40 wt. %, up to 20 wt. % glycol, at least 20% water and glutathione peroxidase mimetic such as but not limited to the compound of formula (I). By means well know in the art, the foregoing compositions are made by vigorous mixing or stirring into a colloid system containing the liposomal vesicles.

The phospholipids of such liposomes can comprise one or more fo the following non-limiting examples: phosphatidylcholine, hydrogenated phosphatidylcholine, phosphatidic acid, phosphatidylserine, phosphatidylethanolamine, phosphatidyglycerol or phosphatidylinositol. Typically soya phospholipids such as Phospholipon 90 (PL-90) is used. The concentration of phospholipid ranges between about 0.5-10% w/w. Cholesterol at concentrations ranging between about 0.1-1% can also be added to the preparation. Examples of alcohols which can be used are: ethanol and isopropyl alcohol. Examples of glycols are propylene glycol and Transcutol™. The source of the phospholipids can be egg, soybean, semi-synthetics, and synthetics. Non ionic surfactants can be combined with the phospholipids in these preparations e.g. PEG-alkyl ethers (Brij-52). Cationic lipids like cocoamides, POE alkyl amines, dodecylamine, cetrimide and like.

The concentration of alcohol (such as ethanol or a C₃ or C₄ alcohol) in the final product ranges from about 20-50%. The concentration of the non-aqueous phase (alcohol and glycol combination) may range between about 22 to 70%. The rest of the carrier contains water and possible additives. For preparation of a formulation for application of such liposomes to the skin, the liposomes can be incorporated in various carriers such as: PVP/VA (gels, membranes, solutions), PVP (gels, membranes, solutions) carbomer gels, polaxomer (gels, solutions), emulsions, creams, Pluronic F127 or Tetronic gels and the like, cellulose derivatives gels, PL-90ant extract gels (aloe vera gel etc), and the like.

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions provided herein, are used in the methods described herein.

Accordingly and in another embodiment, provided herein is a method of treating a neurodegenerative disease in a subject, comprising the step of contacting said subject with a therapeutically effective amount of a composition comprising glutathione peroxidase or its isomer, metabolite, and/or salt therefore and pharmaceutically acceptable carrier or diluent, thereby increasing endogenous levels of anti-oxidant activity. In one embodiment, provided herein is method of inhibiting or suppressing a neurodegenerative disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity, or in another embodiment, a method of reducing the incidence of a neurodegenerative disease in a subject, comprising the step of contacting said subject with a therapeutically effective amount of a composition comprising glutathione peroxidase or its isomer, metabolite, and/or salt therefore and pharmaceutically acceptable carrier or diluent, thereby increasing endogenous levels of anti-oxidant activity.

In one embodiment, the invention provides a method of treating a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity. In another embodiment, the invention provides a method of inhibiting or suppressing a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity, or in yet another embodiment, a method of reducing incidence of a pulmonary disease in a subject, comprising the step of contacting said subject with a therapeutically effective amount of a composition comprising a glutathione peroxidase mimtic or its isomer, metabolite, and/or salt therefore and pharmaceutically acceptable carrier or diluent, thereby increasing endogenous levels of anti-oxidant activity.

In one embodiment, the invention provides a method of treating an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity. In another embodiment, provided herein is a method of inhibiting or suppressing an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity, or in another embodiment, a method of reducing incidence of an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent, thereby increasing endogenous levels of anti-oxidant activity.

In one embodiment, the term “administering” refers to bringing a subject in contact with the compositions provided herein. For example, in one embodiment, the compositions provided herein are suitable for oral administration, whereby bringing the subject in contact with the composition comprises ingesting the compositions. In another embodiment, the compositions provided herein are suitable for topical administration, whereby administering the composition using a patch in another embodiment brings the subject in contact. In one embodiment, the compositions provided herein are in a cream form and are applied directly to the affected area on the subject's skin, thereby bringing the subject in contact with the compositions provided herein. A person skilled in the art would readily recognize that the methods of bringing the subject in contact with the compositions provided herein, will depend on many variables such as, without any intention to limit the modes of administration; the skin condition treated, age, pre-existing conditions, other agents administered to the subject, the severity of symptoms, location of the affected are and the like. In one embodiment, provided herein are embodiments of methods for administering the compounds of the present invention to a subject, through any appropriate route, as will be appreciated by one skilled in the art.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

In one embodiment, the methods provided herein, using the compositions provided herein, further comprise contacting the subject with one or more additional agent.

In one embodiment, provided herein is a method of treating, or inhibiting, suppressing or reducing incidence in other embodiments, of a neurodegenerative disease comprising contacting a subject with a glutathione peroxidase mimetic or its isomer, metabolite, and one or more agent that is carbidopa/levodopa, a selegeline, vitamin E, an amantadine, a pramipexole, a ropinerole, coenzyme Q10, a GDNF, an aldosterone inhibitor. an ACE inhibitor, a probucol analog, tacrine, a heptylphysostigmine, simvastatin, lovastatin, pravastatin, thorvastatin, donepezil, or a combination thereof.

In another embodiment, provided herein is a method of treating, or inhibiting, suppressing or reducing incidence in other embodiments, of a neurodegenerative disease comprising contacting a subject with a glutathione peroxidase mimetic or its isomer, metabolite, whereby contacting is via parenteral administration. In another embodiment, contacting is via topical administration. In another embodiment, contacting is via transdermal administration. In another embodiment, contacting is via intravenous administration. In another embodiment, contacting is via intramuscular administration. In another embodiment, contacting is via subcutaneous administration. In another embodiment, contacting is via bolus injection. In another embodiment, contacting is via sustained release. In another embodiment, contacting is via infusion. In another embodiment, contacting is via cannular administration. In another embodiment, contacting is via intracranial administration. In another embodiment, contacting is via suppositories administration. In another embodiment, contacting is via a combination thereof.

In one embodiment, provided herein is a method of treating, or inhibiting, suppressing or reducing incidence in other embodiments, of a pulmonary disease comprising contacting a subject with a glutathione peroxidase mimetic or its isomer, metabolite, and one or more agent that is a glucocorticoid, a β-2 adrenergic agonist, salmeterol, an anti-cholinergic drug, theophylline, a corticosteroid, a mucolytic agent, an antibiotic, an antiviral, a leukotriene inhibitor or a combination thereof.

In another embodiment, provided herein is a method of treating, or inhibiting, suppressing or reducing incidence in other embodiments, of a pulmonary disease comprising contacting a subject with a glutathione peroxidase mimetic or its isomer, metabolite, whereby contacting is via intravenous administration. In another embodiment, contacting is via intramuscular administration. In another embodiment, contacting is via intraarticular administration. In another embodiment, contacting is via intranasal administration. In another embodiment, contacting is via transnasal administration. In another embodiment, contacting is via parenteral administration. In another embodiment, contacting is via oral administration. In another embodiment, contacting is via aerosolized administration. In another embodiment, contacting is via their combination administration.

In one embodiment, provided herein is a method of treating, or inhibiting, suppressing or reducing incidence in other embodiments, of an inflammatory disease comprising contacting a subject with a glutathione peroxidase mimetic or its isomer, metabolite, and one or more agent that is a tetracycline compound, a nonsteroidal anti-inflammatory drug, a Cox-2 inhibitor, a corticosteroid, S-adenylmethionine, a synovial fluid supplement, a cetyl myristoleate compound, or a combination thereof.

In another embodiment, In another embodiment, provided herein is a method of treating, or inhibiting, suppressing or reducing incidence in other embodiments, of an inflammatory disease comprising contacting a subject with a glutathione peroxidase mimetic or its isomer, metabolite, whereby contacting is via oral administration. In another embodiment, contacting is via intraoral administration. In another embodiment, contacting is via rectal administration. In another embodiment, contacting is via parenteral administration. In another embodiment, contacting is via topical administration. In another embodiment, contacting is via epicutaneous administration. In another embodiment, contacting is via transdermal administration. In another embodiment, contacting is via subcutaneous administration. In another embodiment, contacting is via intramuscular administration. In another embodiment, contacting is via intranasal administration. In another embodiment, contacting is via sublingual administration. In another embodiment, contacting is via buccal administration. In another embodiment, contacting is via intradural administration. In another embodiment, contacting is via intraocular administration. In another embodiment, contacting is via intrarespiratory administration. In another embodiment, contacting is via nasal inhalation administration. In another embodiment, contacting is via a combination administration thereof.

Accordingly and in one embodiment, provided herein is a method of treating Parkinson's disease in a subject, comprising the step of orally contacting the subject with the compound of formula (I) and one or more of Levodopa; Escitalopram; Selegeline, thereby increasing endogenous antioxidant activity, removing free radicals and reducing dyskinesia and associated depression.

In another embodiment, provided herein is a method of treating amyotrophic lateral sclerosis (ALS) in a subject, comprising the step of parenterally contacting the subject with the compositions provided herein and one or more of Riluzole, ceftriaxone, coenzyme QIO, memantine or sodium phenylbutyrate, thereby increasing endogenous antioxidant activity, removing free radicals and reducing glutamate excitotoxicity.

In one embodiment, provided herein is a method of treating cystic fibrosis (CF) disease in a subject, comprising the step of contacting the subject with the compositions described herein in an aerosolized form and one or more of domase alfa, tobramycin or their combination thereby increasing endogenous antioxidant activity, removing free radicals providing a mucolytic agent and removing pathogenic bacteria.

In another embodiment, provided herein is a method of treating acute respiratory distress syndrome (ARDS) in a subject, comprising the step of intranasally contacting the subject with the compound of formula (I) and one or more of Sivelestat, salbutamol or their combination, thereby increasing endogenous antioxidant activity, removing free radicals providing O₂ agonists and inhibiting neutrophil elastate.

in one embodiment, the invention provides a method of treating rheumatoid arthritis (RA) in a subject, comprising the step of topically contacting the subject with the compositions provided herein and one or more of fentanyl, ibuprofen, triamcinolone hexacetonide (TH) or their combination, thereby increasing endogenous antioxidant activity, removing free radicals and, providing an analgesic and non-steroidal anti inflammatories.

A person skilled in the art would readily recognize that combination therapy as described in the methods and compositions provided herein, could be administered eithe simultaneously or consecutively and so long as they are administered for the same condition, would be encompassed herein.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1-42. (canceled)

43-58. (canceled)

59. A method of treating, inhibiting, suppressing or reducing incidence of a neurodegenerative disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

60. A method of treating, inhibiting, suppressing or reducing incidence of a pulmonary disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

61. A method of treating, inhibiting, suppressing or reducing incidence of an inflammatory disease in a subject, comprising the step of contacting said subject with a therapeutically effective amount of a composition comprising a glutathione peroxidase mimetic or its isomer, metabolite, and/or salt therefore and pharmaceutically acceptable carrier or diluent.

62. The method of claim 59, wherein the neurodegenerative disease is Parkinsons disease (PD), Alzheimers disease (AD), Huntingtons disease (HD), amyotrophic lateral sclerosis (ALS), a triplet repeat disease (ARD), Friedreich's ataxia, stroke, multi-infarct; dementia, multiple sclerosis, chronic fatigue syndrome, or a combination thereof.

63. The method of claim 60, wherein the pulmonary disease is chronic obstructive pulmonary disease (COPD), cystic fibrosis (CF), dyspnea, emphysema, wheezing, pulmonary hypertension, pulmonary fibrosis, asthma, hyper-responsive airways, pulmonary bronchoconstriction, respiratory tract inflammation or allergies, chronic bronchitis, bronchoconstriction, acute respiratory distress syndrome (ARDS), reactive airway disease, infantile respiratory distress syndrome (infantile RDS), pain, allergic rhinitis, lung cancer, chronic bronchitis or a combination thereof.

64. The method of claim 61, wherein the inflammatory disease is rheumatoid arthritis, Behcet's disease, polyarteritis nodosa, Wegener granulomatosis, lupus, Takaysu's arteritis, osteoarthritis, juvenile idiopathic arthritis, alcoholic cirrhosis or a combination thereof.

65. The method of any one of claims 59-61, wherein said glutathione peroxidase mimetic is an organoselenium compound, ebselen or their combination.

66. The method of claim 65, wherein said organoselenium compound is benzisoselen-azoline or -azine derivatives represented by the compound of formula I:

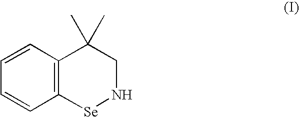

67. The method of claim 65, wherein said organoselenium compound is benzisoselen-azoline or -azine derivatives represented by the following general formula II: 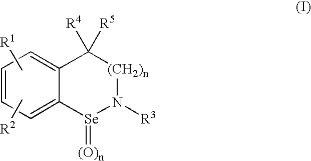
wherein
R¹=R²-hydrogen; lower alkyl; OR⁶; —(CH₂)ₘNR⁶R⁷; —(CH₂)_{q}NH₃; —(CH₂)ₘ NHSO₂ (CH₂)₂ NH₂; —NO₂; —CN; —SO₃H; —N⁺(R⁵)₂ O⁻; F; Cl; Br; I; —(CH₂)ₘ R⁸; —(CH₂)ₘ COR⁸; —S(O)NR⁶ R⁷; —SO₂ NR⁷; —CO(CH₂p COR⁸; R⁹;
R³=hydrogen; lower alkyl; aralkyl; substituted aralkyl; —(CH₂), COR⁸; —(CH₂)_{q}R⁸; —CO(CH₂)ₚ COR⁸; —(CH₂)ₘ SO₂ R⁸; —(CH₂)ₘ S(O)R⁸;
R⁴=lower alkyl; aralkyl; substituted aralkyl; —(CH₂)ₚ COR⁸; —(CH₂)ₚR⁸; F;
R⁵-lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸−lower alkyl; aralkyl; substituted aralkyl; aryl; substituted aryl; heteroaryl; substituted heteroaryl; hydroxy lower alkoxy;
R⁹ is represented by any structure of the following formulae: 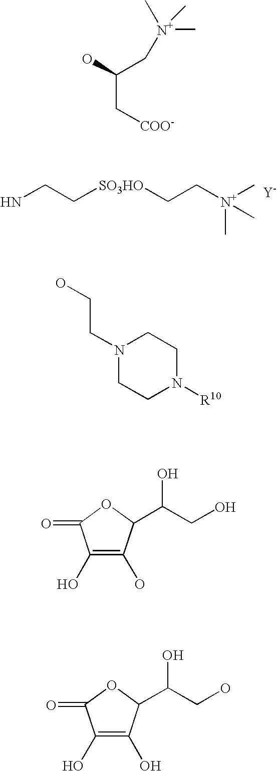
R¹⁰ hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

68. The method of claim 65 where the organoselenium compound is represented by the compound of formula (XI): 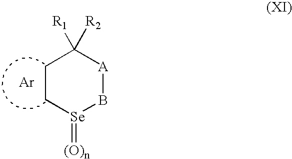
in which: R₁-hydrogen; lower alkyl; optionally substituted aryl; optionally substituted lower aralkyl; R₂-hydrogen; lower alkyl; optionally substituted aryl; optionally substituted lower aralkyl; A=CO; (CR₃R₄)ₘ; B—NR₅; O; S; Ar=optionally substituted phenyl or an optionally substituted radical of formula: 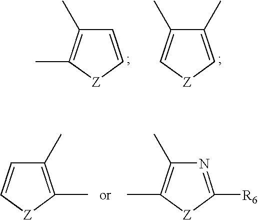
in which: Z=O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂(aryl); R₆-hydrogen; lower alkyl; optionally substituted aryl; optionally substituted lower aralkyl; optionally substituted heteroaryl; optionally substituted lower heteroaralkyl; trifluoromethyl; 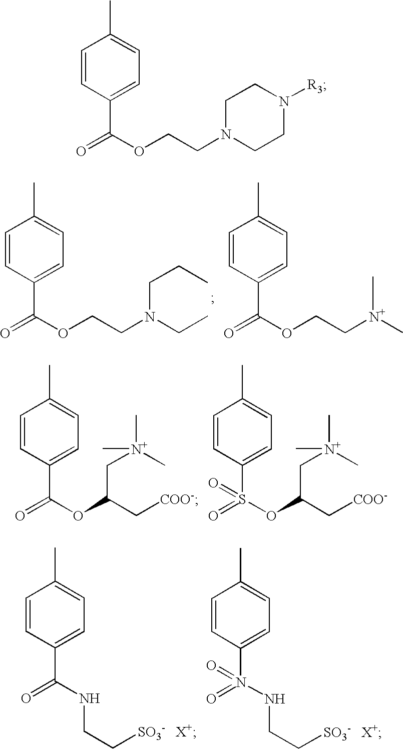
m=0 or 1; n=0 or 1; X⁺ represents the cation of a pharmaceutically acceptable base; and their pharmaceutically acceptable salts of acids or bases.

69. The method of claim 65, wherein the organoselenium compound comprises 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, or 4,4-dimethyl-thieno-[2,3-e]-isoselenazine-1-oxide.

70. The method of claim 65 wherein the organoselenium compound is represented by the compound of formula (XII): 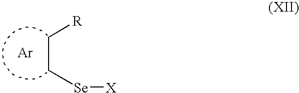
in which:
R=hydrogen; —C(R₁R₂)-A-B;
R₁=lower alkyl; optionally substituted aryl; optionally substituted lower aralkyl;
R₂=lower alkyl; optionally substituted aryl, optionally substituted lower aralkyl;
A=CO; (CR₃R₄)ₙ;
B represents NR₅R₆; N⁺R₅R₆R₇Y⁻; OR₅; SR₅;
Ar=an optionally substituted phenyl group or an optionally substituted radical of 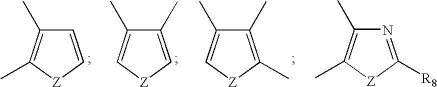
in which Z represents O; S; NR₅; when R=—C(R₁R₂)-A-B or Ar=a radical of formula 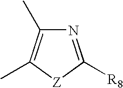
in which Z=O; S; NR₅; when R is hydrogen; X—Ar(R)—Se—; —S-gluththione; —S—N-acetylcysteine; —S-cysteine; —S-penicillamine; —S-albumin; —S-glucose; 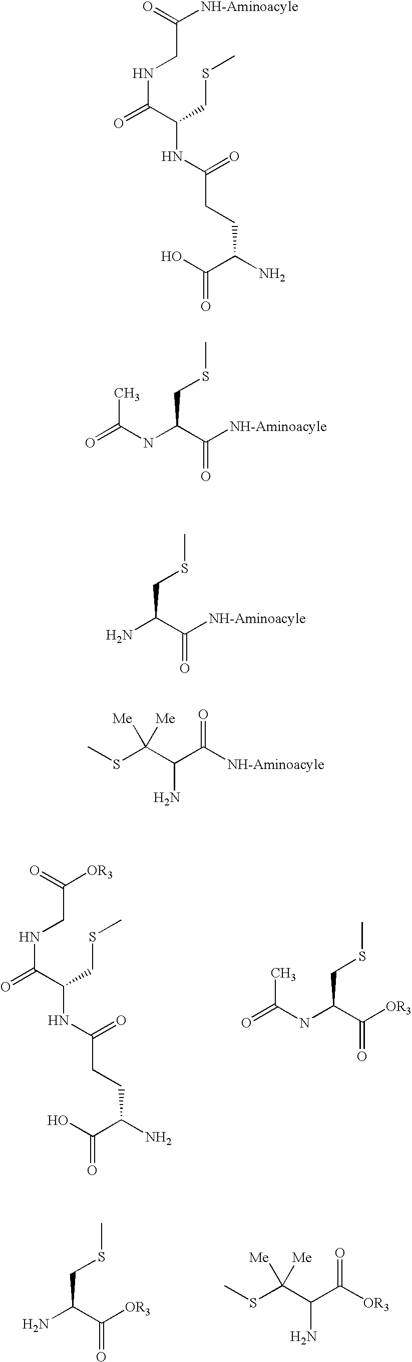
R₃=hydrogen; lower alkyl; optionally substituted aryl, optionally substituted lower aralkyl;
R₄=hydrogen; lower alkyl; optionally substituted aryl: optionally substituted lower aralkyl;
R₅=hydrogen; lower alkyl; optionally substituted aryl: optionally substituted lower aralkyl; optionally substituted heteroaryl; optionally substituted lower heteroaralkyl; CO(lower alkyl); CO(aryl); SO₂(lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl; optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl, 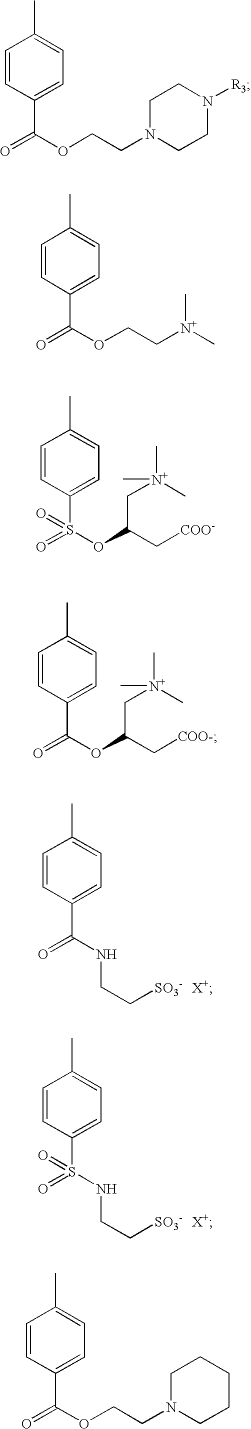
n=0 or 1; X⁺ represents the cation of a pharmaceutically acceptable base;
Y⁻ represents the anion of a pharmaceutically acceptable acid;
and their salts of pharmaceutically acceptable acids or bases.

71. The method of claim 70 wherein the compound comprises di[2-[2′-(1′-amino-2′-methyl)propyl]phenyl]diselenide; di[2-[2′-(1′-amino-2′-methyl)propyl]phenyl]-diselenide dihydrochloride; di[2-[2′-(1′-ammonium-2′-methyl)propyl]phenyl]diselenide di-paratoluenesulphonate; di[2-[2′-(1′-amino-2′-methyl)propyl]-4-methoxy]phenyl-diselenide; di[2-[2′-(1′-methylamino-2′-methyl)propyl]phenyl]-diselenide; di[2-[2′-(1′-methylamino-2′-methyl)propyl]-phenyl]-diselenide dihydrochloride; di[2-[2′-(1′-dimethylamino-2′-methyl)propyl]phenyl]-diselenide; di[2-[2′-(1′-trimethylammonium-2-methyl)propyl]phenyl]-diselenide di-paratoluenesulphonate; S-(N-acetyl-L-cysteinyl)-[2-[2′-(1′-amino-2′-methyl)-propyl]phenyl]-selenide; or S-glutathionyl-[2-[2′-(1′-amino-2′-methyl)-propyl]-phenyl]-selenide.
